(19) United States
(12) Patent Application Publication
(10) Pub. No.: US 2004/0149688 A1
(43) Pub. Date: Aug. 5, 2004

(54) Method for producing a biomimetic membrane, biomimetic membrane and its applications

(75) Inventors: Alexandra Fuchs, Chevaloi de Voreppe (FR); Michel Heitzmann, Crolles (FR); Francoise Vinet, Grenoble (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: COMMISSARIAT A L'ENERGIE ATOMIQUE, PARIS (FR)

(21) Appl. No.: 10/667,785

(22) Filed: Sep. 23, 2003

(30) Foreign Application Priority Data
Sep. 24, 2002 (FR) 02 11770

(51) Int. Cl. A61L 2/00; B05D 5/00

(52) U.S. Cl. 216/56; 427/2.1; 427/258; 427/271

(57) ABSTRACT

The invention concerns a method for producing a biomimetic membrane, a biomimetic membrane and the applications of said membrane.

Said biomimetic membrane (10) has one or several through pores (24) and is characterised in that it is formed of at least two different micro-machinable materials one of which forms the wall (23) of said pore(s), whereas the other or others of said materials form(s) the remainder of said membrane.

Applications: performing studies on the dynamic and functional properties of biological membranes; manufacture of biocatalysis microsystems and detection or dosing of substances.

### TECHNICAL FIELD

The present invention concerns a method for producing a biomimetic membrane, a biomimetic membrane and the applications of said membrane.

More precisely, said method makes it possible to produce a stable porous membrane, the structure of which mimics the structural characteristics of biological membranes and which, moreover, has zones provided with specific physical/chemical properties, in the image of biological membranes.

Said type of membrane is capable of serving as an experimental model in all research work concerning the dynamic and functional properties of biological membranes, and especially for studying the mechanisms of the trans-membrane transport of molecules and the membrane potential, diffusion, reaction and interaction processes at biological interfaces, the functional dynamic of isolated membrane proteins, membrane ligand/receptor recognition and affinity reactions or even the construction of macromolecular architectures on biological interfaces, said type of studies being of direct interest to industry and health, and in particular the pharmaceuticals industry.

Furthermore, said membrane is also capable of being used in the manufacture of biocatalysis Microsystems and microsystems for the detection or dosing of substances and being used, in particular, as sensitive layers in biosensors.

### STATE OF THE PRIOR ART

Despite the considerable advances made over that last few decades in cellular biology, biological membranes remain a major area of study both as regards their constitution and their functioning.

The experimental membrane models currently used are, for the most part, formed of phospholipidic bi-layers such as phosphatidyl choline, phosphatidyl ethanolamine or phosphatidyl serine bi-layers, within which are integrated proteins, which act as ducts, such as λ-hemolysine.

Although said type of experimental models enable the insertion and functioning mode of membrane proteins to be studied, they do not on the other hand include one of the key functions of biological membranes, which is to assure exchanges between the intra- and extra-cellular media.

Furthermore, lipidic bi-layers have the drawback of being unstable and being not very easy to handle. As for the proteins playing the role of ducts in said bi-layers, they are labile and their diameter cannot be adjusted.

Consequently, there is considerable interest in replacing lipidic bi-layers constituting biological membranes by artificial interfaces that are capable of housing functional proteins and/or providing permeability characteristics similar to that of biological membranes.

The possibilities of miniaturisation offered by the technologies for making electronic microsystems have led a certain number of teams to envisage using said technologies for producing artificial membranes. However, the very limited number of publications in this field bears witness to the difficulties posed in producing said membranes.

The most avant-garde artificial membrane made to date is without doubt that produced by LI et al. (Nature, 412, 166-169, 2001 (1)).

Said membrane is formed of silicon nitride (Si₃N₄) and comprises a single pore of 1 to 5 nm diameter. It is produced by a method that consists in:

depositing a layer of Si₃N₄ on the front face of a silicon substrate by a low pressure chemical vapour deposition (LP-CVP);

partially liberating said layer by a lithography and a wet etching of the rear face of the substrate;

creating, within the part of the layer of Si₃N₄, thus liberated, a non-transversal cavity by means of a focused ion beam, the base of said cavity being situated on the side of the front face of said layer;

levelling the front face of said layer by the application of an argon ion beam up to the point where the base of said cavity is pierced, said piercing thereby forming a pore of around 100 nm diameter, and;

partially altering said pore by the argon ion beam under critical experimental conditions that are difficult to control.

Apart from the fact that the process developed by these Authors requires—as they themselves recognise—carrying out numerous complementary studies before its use can be envisaged on an industrial scale, it turns out that it has the major disadvantage of leading to the formation of a pore in which the wall is necessarily formed of the same material as that which forms the remainder of the membrane.

Furthermore, the feedback loop that makes it possible to control the diameter of the pore only allows the formation of a single pore at each time.

However, in numerous fields, it is indispensable to have at one's disposal artificial membranes that have a matrix of calibrated pores in which the wall is provided with functionalities different to those of the non-porous portions of the faces of said membranes, which implies that the wall of the pores must be able to be formed out of different materials to those used to form the remainder of the membranes.

In addition, International patent application WO 01/36321 (2) describes a method for producing an artificial membrane comprising a plurality of nanopores of diameter less than 50 nm and which is intended to be used to filter biological substances.

Said method comprises, in its most simple embodiment:

depositing, on the front face of a substrate (for example, of silicon), a sacrificial etching boundary layer (for example, of Si₃N₄), by a low pressure chemical vapour deposition;

depositing, on said sacrificial etching boundary layer, a structural layer (for example, of polycrystalline silicon or Si₃N₄);

forming, within the structural layer, holes with a width of around one micron, by an etching of said layer by means of a chlorine plasma after thermal growth of a silicon oxide layer (SiO₂), said holes being intended to define the shape of the future pores of the membrane;

thermally growing, on the structural layer, a sacrificial layer of SiO₂, the thickness of which defines the nanometric dimensions of the pores;

forming, within the sacrificial SiO₂ layer, specific anchoring points to ensure a mechanical liaison between said layer and the layer intended to cover it in the following step, said anchoring points being formed by a partial etching of said sacrificial layer of SiO₂ on the surface and in the interior of the pores;

depositing, on said sacrificial layer of SiO₂, a “plug” layer of polycrystalline silicon, the thickness of which is chosen in such a way that the deposition of said layer fills the pores (a 1.5 μm);

planing down the “plug” layer of polycrystalline silicon up to the point where the polycrystalline silicon only remains in the pores;

polishing the residue of the “plug” layer of polycrystalline silicon;

depositing, on each face of the edifice thus formed, a protective layer (for example of Si₃N₄);

eliminating a part of said protective layer situated on the rear face of the substrate, then eliminating the substrate thus laid bare and the etching boundary layer, by chemical etching (KOH), and;

eliminating the sacrificial etching boundary layer and the sacrificial layer of SiO₂ by chemical etching (HF or SF₆) combined with oxygen plasma etching.

Thus, in its simplest embodiment, said method comprises no less than eleven steps—which makes it a very costly method—, and leads to a membrane being obtained which is formed by the structural layer (polycrystalline silicon or Si₃N₄) and in which the wall of the pores that cross through it is not homogeneous since said wall is partially formed of the material of the structural layer and of the polysilicon of the “plug” layer.

Although these characteristics do not constitute an obstacle to the use of said membrane for the filtration of biological substances, they do however prevent its use as an experimental model in studies concerning biological membranes.

The Inventors therefore set themselves the aim of providing a method that makes it possible to produce an artificial membrane suited to reproducing the structural and functional characteristics of biological membranes, while at the same time being very simple to use, in such a way that the production costs for said membrane are as low as possible.

### DESCRIPTION OF THE INVENTION

This aim is achieved by the present invention which proposes a method for producing a biomimetic membrane, which is characterised in that it comprises the following steps:

a) depositing, on at least one of the principal faces of a plate A of a micro-machinable material, a layer B comprising one or several strata each formed of a micro-machinable material,

b) forming one or several through holes within layer B, each hole having a wall formed of the material(s) of said layer B and a bottom formed of the material of plate A,

c) depositing, on said layer B, the wall and the bottom of each hole, a layer C of a micro-machinable material, which closely hugs the wall and the bottom of said hole,

d) eliminating layer C from the underlying face of layer B and, at the centre of each hole, from the underlying face of plate A, while at the same time leaving a residue of layer C on the wall of said hole, said residue delimiting a pore in which the wall is formed of the material of layer C and in which the bottom is formed of the material of plate A, and

e) liberating at least a part of the B in which are found one or several pores formed from step d), by the partial or total elimination of plate A.

One thus obtains a membrane which is formed of the material(s) forming layer B except at the level of the wall of the pore(s) that it comprises, said wall being, in fact, formed of the material of layer C.

Therefore, depending on whether one decides to form layer B in one or several strata and to use identical or different micro-machinable materials to form said stratum or strata on the one hand, and layer C on the other hand, it is possible to obtain, by the method according to the invention, different types of porous membrane, and in particular:

membranes in which the wall of the pore(s) that they contain is formed of the same material as that forming the portions of the two principal faces of said membranes which are not occupied by said wall (for example, if layer B only comprises a single stratum formed of the same material as that used to form layer C);

membranes in which the wall of the pore(s) that they contain is formed of a different material to that forming the portions of the two principal faces of said membranes which are not occupied by said wall (for example, if layer B only comprises a single stratum formed of the same material as that used to form layer C);

membranes in which the wall of the pore(s) that they contain is formed of a different material to that forming the portions of the two principal faces of said membranes which are not occupied by said wall, said portions being, in fact, themselves formed of a material different from one face to the other of said membranes (for example, if layer B comprises two strata formed of two different materials, not only to each other but also to the material used to form layer C).

As mentioned above, the method according to the invention comprises, as a first step, or step a), depositing, on one of the principal faces of a plate A formed of a micro-machinable material, a layer B comprising one or several strata each formed of a micro-machinable material, which may be identical or different from one stratum to the next.

The term “micro-machinable material” herein designates any material that is suitable for the techniques used in the manufacture of microstructures and nanostructures, and in particular to thin film deposition, lithography and etching techniques.

Said type of material may in particular be of silicon, polycrystalline silicon (or polysilicon), silica, an oxide such as silicon oxide or titanium oxide, a nitride such as silicon nitride or aluminium nitride, a metal such as gold, copper or nickel, a metal alloy such as an aluminium alloy or a copper/nickel alloy, a glass such as borosilicate glass or phosphosilicate glass, a ceramic such as a silicon carbide, a polymer such as a polyimide or even a sol-gel.

According to the invention, the deposition, on layer A, of the stratum or strata forming layer B may be achieved by any of the techniques used for forming thin films, providing they are adapted to the materials chosen to form said layer B and plate A that said layer is intended to cover, or by a combination of these techniques.

Thus, for example, said deposition may be achieved by thermal oxidation, by chemical vapour deposition (CVD), by low pressure chemical vapour deposition (LP-CVD), by thermal evaporation, by electrolytic or electrochemical deposition, by cathode sputtering or even by means of a pulsed laser.

Whatever the number of strata that it comprises and the technique chosen for depositing them, layer B has a thickness that corresponds to the thickness that one wishes to give to the membrane, which is, preferably, between 5 nm and 5 μm as a function of the purpose of said membrane.

After the deposition of layer B, the method according to the invention comprises a step, or step b), which consists in forming one or several through holes within said layer. Each hole obtained thereof thus has a wall formed by the material(s) of layer B and a bottom formed of the material of plate A.

The method according to the invention is intended to produce a membrane having, either a single pore, or a plurality of pores that can reach 100 million pores per mm² of surface area, and typically up to 20 million pores per mm² of surface area.

Thus, the number of through holes formed within layer B is between 1 single hole (for the whole surface of said layer) and 100 million holes per mm² of surface area of layer B and, preferably, between 1 single hole and 20 million holes per mm² of surface area of layer B.

Considering the aforementioned and in order to make the description as simple as possible, it is considered, hereafter, that layer B and the membrane respectively comprise holes and pores and not a single hole or a single pore.

Preferably, the holes formed in layer B are substantially cylindrical and are formed by lithography, for example DUV (“deep-ultraviolet”) or electron beam type lithography, followed by an etching.

Although said etching may be carried out either by wet or dry means, preferentially dry etching is used in so far as it makes it possible, firstly, to obtain holes of smaller-cross-section that those that would be obtained by wet etching and, secondly, to control more fully the cross-section of said holes—which is preferably between 10 nm and 1 μm in the case of cylindrical holes—as well as their verticality.

The third step of the method according to the invention, or step c), consists in depositing, on layer B, the wall and the bottom of the holes, a layer C of a micro-machinable material, which closely hugs the wall and the bottom of said holes, and in which the face opposite to that situated in contact with layer B, the wall and the bottom of the holes, consequently delimits a depression in the form of a U within said holes.

The thickness of layer C should be substantially constant and less than half of the smallest dimension presented by the cross-section of the holes and at least equal to the thickness of layer B. Thus, in the case of substantially cylindrical holes, the thickness of layer C must be less than their radius.

Here again, the deposition of layer C may be achieved by any of the techniques used for forming thin films, providing it is suited to the materials chosen to form said layer and the underlying layer B.

Once layer C has been deposited, the method according to the invention provides for, in a step d), eliminating said layer from the underlying face of layer B and, in the centre of the holes, from the underling face of layer A, while at the same time conserving a residue of layer C on the wall of the holes, in such a way that each residue delimits a pore in which the wall is formed of the material of layer C and in which the bottom is formed of the material of plate A.

This may be achieved by an anisotropic etching of layer C, in other words by a dry etching, which one carries out perpendicularly to the principal plane of said layer and which makes it possible to avoid the erosion of the parts of said layer that cover the wall of the holes.

This results in the formation, in each hole, of a residue of layer C or “spacer” which covers the totality of the wall of said hole, and which delimits an opening, said opening corresponding to a pore of the membrane being produced.

Thus, steps c) and d) of the method according to the invention make it possible to adjust, while reducing it, the cross-section of the holes formed in layer B during step b), to that which must be possessed by the pores of the membrane that one wishes to produce.

Furthermore, in choosing for layer C a material different to that or those that form layer B, the steps c) and d) make it possible to confer to the membrane pores in which the wall is chemically different to the remainder of said membrane.

It is thus possible, by using to form either layers B and C, a hydrophilic type material such as silica, and for the other of said layers, a hydrophobic type material such as silicon, and by functionalising if appropriate said materials differently, for example by grafting of chemical or biochemical compounds, manufacturing a membrane provided both with sites with hydrophobic character and sites with hydrophilic character and having different ion charge densities.

In accordance with the invention, the anisotropic etching of layer C is preferentially a reactive ion etching, which may be carried out by means of an RIE (reactive ion etching) equipment, MRIE (magnetically enhanced RIE), TCP (transformer coupled plasma), ICP (inductively coupled plasma), DPS, ECR, DECR or analogous.

The method according to the invention then comprises a step, or step e), which consists in liberating at least a part of layer B in which are found the pores formed in the previous step, by partial or total elimination of plate A.

In a first preferred embodiment of the method according to the invention, step e) comprises the total elimination of plate A in such a way that layer B is completely liberated of it.

One thus obtains a membrane that is in the form of a sheet, which may be of circular shape, parallelepiped or other. Said type of membrane is in particular suited to being used in forming detection and substance Microsystems, and in particular to being used as thin films in biosensors.

In another preferred embodiment of the method according to the invention, step e) comprises the following steps:

e₁) fastening, on the free face of layer B, a plate A′ of a micro-machinable material, and

e₂) hollowing out plates A and A′ so as to liberate the part of layer B in which are found the pores, while at the same time leaving the edges of said plates as well as a part of their face opposite to that situated in contact with said layer B.

One thus obtains a membrane which is integral with two chambers, which are arranged on either side of said membrane and which are provided with an opening in such a way that the interior of said chambers is accessible.

Said chambers could in particular be filled with solutions of polymeric gels or not, miming the intra- or extra-cellular media in such a way as to place the membrane in conditions comparable to physiological conditions, or could be used as reaction chambers.

Said type of membrane is therefore particularly well suited to carrying out studies on biological membranes, and especially on diffusion, reaction and interaction processes at the interfaces or on the functional dynamic of the membrane proteins.

Preferably, plates A and A′ are formed of the same material and are covered, on their principal phase opposite to that situated in contact with layer B, with a layer D of a micro-machinable material.

Under these conditions, step e₂) of the method according to the invention preferably comprises:

a lithography followed by a wet or dry etching o partially eliminate layers D,

a wet etching to hollow out plates A and A′ while at the same time leaving a residue of said plates which covers layer B, and

a dry etching to liberate the part of layer B in which are found the pores.

In accordance with the invention, layer B comprises, preferably, one or two-strata.

When layer B comprises a single stratum, then said stratum is, preferentially, formed of a micro-machinable material different to that forming layer C.

When layer B comprises two strata, then said strata are, preferentially, formed of different materials, both to each other but also to the material forming layer C.

In all cases, the materials forming plates A and A′, layer B and layer C are, preferably, chosen from among silicon, polycrystalline silicon, silica, silicon oxide and silicon nitride.

In a particularly preferred embodiment of the method according to the invention, this further comprises, after step e), a step of functionalising the wall of the pores and/or the portions of the principal faces of the membrane that are not occupied by said wall.

In the present invention, “functionalisation” of a material is taken to mean any treatment whose purpose is to modify the physical/chemical properties or to confer it with specific physical/chemical properties.

Said functionalisation, which is chosen as a function of the purpose of the membrane, may in particular consist in a pre-treatment such as hydroxylation (by chemical means or by oxygen plasma), followed by a liquid phase silanisation that makes it possible to render the treated zone hydrophobic or, quite the reverse, hydrophilic (depending on the type of silane used), or to confer to said zone chemical functions (diols, aldehydes, etc.) suited to being used subsequently for the grafting of interesting molecules, in particular biological molecules (polypeptides, proteins, oligonucleotides, fragments of DNA or RNA, etc.).

It should be noted that all of said treatments are known in themselves. They are in particular described by MARTIN and GROVE in Biochemical Microdevices, 3(2), 97-108, 2001 (3).

The silane based functionalisation has the advantage of leading to the formation, on the treated zone, of monomolecular layers of around 2 to 4 nm and, consequently, not significantly modifying the thickness of the membrane.

Preferably, the functionalisation step comprises a functionalisation of the wall of the pores and a functionalisation of the portions of the principal faces of the membrane which are not occupied by said wall, said functionalisations being different.

Said type of differential functionalisation makes possible an advantageous modulation of the physical/chemical environments at the interface, suited to further strengthening the biomimetic character of said membrane. Indeed, it is thus possible to locally form zones having specific physical/chemical properties while at the same time maintaining homogeneity of the whole (thickness, pore diameter, overall chemical structure).

Moreover, it offers the possibility of incorporating membrane proteins in the pores of the membrane, particularly by the LANGMUIR-BLODGETT technique—which makes it possible to prepare, with a strict control of the thickness and the molecular organisation, lamellar type lipidic stacking, by transfer on solid substrate of a monomolecular film formed at the air-water interface—while playing on a more hydrophilic functionalisation of the wall of the pores, suited to retaining the hydrophilic part of the extra-membrane zones of the proteins.

It is thus possible to deposit, on each of the faces of the membrane, a lipidic bi-layer which covers said face and the wall of the pores and to form by appropriately choosing the amphiphilic molecules used for forming the LANGMUIR-BLODGETT layers, a controlled and oriented incorporation of proteins in such a way that they are found in a lipidic environment associated with a structure whose thickness and porosity characteristics are similar to those of a biological membrane.

A further aim of the invention is a biomimetic membrane that has one or several through pores and which is characterised in that it is formed of at least two different micro-machinable materials, one of which forms the wall of said pore(s), whereas the other(s) material(s) form the remainder of said membrane.

According to a first advantageous embodiment of said membrane, it has a surface area between 1 μm² and 1 cm² and a thickness between around 5 nm and 5 μm.

According to a further advantageous embodiment of said membrane, it has 1 single pore or a plurality of pores that can reach 100 million pores per mm² of surface area and, preferably, from 1 pore to 20 million pores per mm² of surface area.

According to yet a further advantageous embodiment of said membrane, the pore(s) of said membrane are substantially cylindrical and have a diameter between 5 and 500 nm.

Preferably, said membrane is formed of two or three different micro-machinable materials.

When said membrane is formed of two micro-machinable materials, then one forms the wall of the pore(s) that it comprises, whereas the other forms the remainder of said membrane.

When said membrane is formed of three micro-machinable materials, then, preferably, one forms the wall of the pore(s) that it comprises, another forms the portions of one of the principal faces of said membrane which are not occupied by said wall, and the final material forms the portions of the other of the principal faces of said membrane which are not occupied by said wall.

In all cases, the micro-machinable materials that form the membrane are, preferably, chosen from among silicon, polycrystalline silicon, silica, silicon oxide and silicon nitride.

According to a preferred embodiment of said membrane, it is integral with two chambers that are arranged on either side of said membrane, which have a base, a lateral wall and a wall opposite said base, and in which the base is formed of said membrane, whereas their wall opposite said base is provided with an opening.

Preferably, the lateral wall of the chambers and the wall of said chambers that is opposite to their base are formed of a micro-machinable material, which is also preferentially chosen from among silicon, polycrystalline silicon, silica, silicon oxide and silicon nitride.

According to a particularly preferred embodiment of said membrane, the wall of the pore(s) that it comprises bears chemical and/or biochemical functions different to those borne by the portions of the principal faces of said membrane which are not occupied by said wall.

The method according to the invention has numerous advantages.

In particular, it makes it possible to produce “made to measure” biomimetic membranes, in other words membranes which have structural and functional characteristics exactly suited to the use for which said membranes are intended, particularly in that it offers the possibility:

of modulating the thickness of said membrane as well as the size and density of the pores that it comprises, over dimension ranges going from nanometres to micrometers,

of also modulating the physical/chemical properties (hydrophilic/hydrophobic, ion charges, etc.) of the materials forming the wall of the pores and the remainder of the membrane,

of grafting onto said materials interesting molecules, and in particular biological molecules (polypeptides, proteins, oligonucleotides, fragments of DNA or RNA, etc.),

of forming pores of the size of a membrane protein, suited to housing said protein, which renders said membranes particularly interesting for carrying out studies on isolated proteins,

of producing membranes provided with accessible chambers, which considerably simplifies their use in carrying out experimental studies.

In addition to all of the aforementioned advantages, it has the advantage of being simple to use and, consequently, is economically interesting.

A further aim of the invention is the application of a membrane as defined hereabove to performing studies on the dynamic and functional properties of biological membranes.

A yet further aim of the invention is the application of said membrane to the production of biocatalysis microsystems or the detection or dosing of substances.

The possibility of forming the membrane according to the invention in very limited thicknesses, particularly in the range from several nm to several tens of nanometres, makes it possible to increase the diffusion rate through said membrane compared to that observed with a conventional polymeric membrane.

Consequently, certain operations (dialysis, transport, etc.) which are performed in several hours, or even several days, with a polymeric membrane may be performed in several seconds with the membrane according to the invention.

In addition to the aforementioned provisions, the invention comprises other provisions that will become clear on reading the following additional description, which relates to two examples of embodiments of the method according to the invention as well as to one example of a membrane produced by said method, and which is given by way of illustration and in no-wise limitative, in referring to the appended drawings.

### BRIEF DESCRIPTION OF THE DRAWINGS

FIGS. 1 to 5 schematically illustrate, in cross-sectional form, the respective steps a) to e) of the method according to the invention, in a first embodiment of said method.

FIGS. 6 to 9 schematically illustrate, in cross-sectional form, step e) of the method according to the invention in a second embodiment of said method.

FIG. 10 corresponds to two photographs taken with an optical microscope, at two different enlargements (1000× and 200×), of a membrane as obtained at the end of step e) of the method according to the invention.

### DETAILED DESCRIPTION

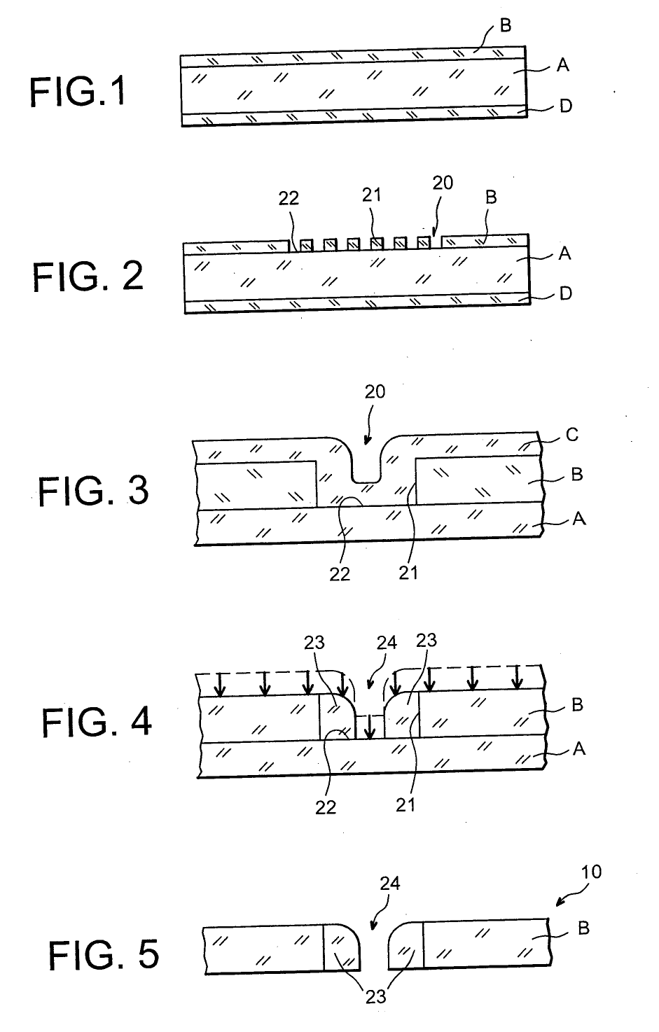

We will refer firstly to FIGS. 1 to 4 which schematically represent the respective steps a) to e) of the method according to the invention, in a first embodiment of said method.

As can be seen in FIG. 1, the first step of the method, or step a), comprises depositing, on the two principal faces of a plate A formed for example of silicon, two layers, respectively, B and D, formed for example of thermal silicon oxide.

The second step of the method, or step b), which is illustrated in FIG. 2, consists in forming within layer B, by a lithography followed by a dry etching of said layer, for example by fluorinated or chlorinated chemistry, a plurality of holes 20 that extend right through said layer.

The wall 21 of holes 20 thus produced is therefore formed of the material of layer B, whereas their bottom 22 is formed of the material of plate A.

The following step, or step c), consists, as is shown in FIG. 3, in depositing, on layer B, the wall 21 and the bottom 22 of the holes 20, a layer C formed either of SiO₂ like layer B, or of another micro-machinable material such as silicon nitride or polycrystalline silicon.

Said layer C has a substantially constant thickness, which is less than half of the smallest dimension of the cross-section of the holes 20 as well as the thickness of layer B, in such as way that its face opposite that situated in contact with layer B, wall 21 and bottom 22 of said holes delimits a depression in the form of a U within said holes.

Then, in step d), which is illustrated in FIG. 4, one eliminates, by anisotropic etching (symbolised by arrows in the Figure), layer C until the underlying face of layer B is reached and, in the central part of the holes 20, the underlying face of plate A. Said etching leads to the formation, in each hole 20, of a residue 23 of layer C or “spacer”, which covers the wall 21 of said hole and which thus delimits an opening corresponding to a pore 24 of the membrane being produced.

As can be seen in FIG. 5, which shows step e), the elimination, by a dry or wet etching, of the plate A makes it possible to liberate layer B and thus obtain a porous membrane 10 which is in the form of a sheet and which is formed of the material of layer B except at the level of the wall of the pores 24 that it comprises where it is formed of the material of layer C.

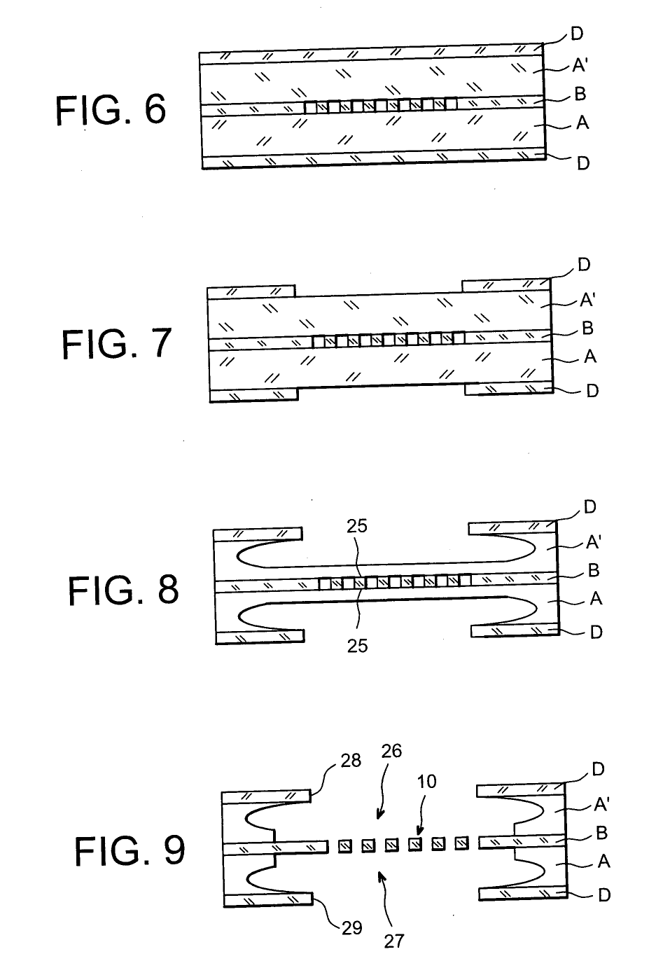

We will now refer to FIGS. 6 to 9 which schematically illustrate step e) of the method according to the invention in a second embodiment of said method that makes it possible to produce a membrane provided with accessible chambers.

As can be seen in FIG. 6, step e) firstly comprises fastening, for example by molecular bonding, a plate A′ on the free face of layer B, said plate A′ being covered, on its face opposite to that in contact with layer B, a layer D.

In the embodiment illustrated in FIG. 6, the plate A′ and the layers D are respectively formed of the same materials as those forming plate A and layer B.

However, it can be otherwise.

As shown in FIG. 7, the part of layers D, which is situated opposite the part of layer B in which are found the pores 20 is then eliminated by a lithography followed by a wet or dry etching.

Then, a wet etching makes it possible to hollow out the plates A and A′, while at the same time conserving a residue 25 of said plates on each of the faces of layer B (FIG. 8), said residue being secondarily eliminated by a dry etching, for example by means of a chlorinated plasma (FIG. 9).

One thus obtains a membrane 10 that is integral with two chambers, respectively 26 and 27, which are arranged on either side of said membrane and which are provided with an opening, respectively 28 and 29, making it possible to access the interior of said chambers.

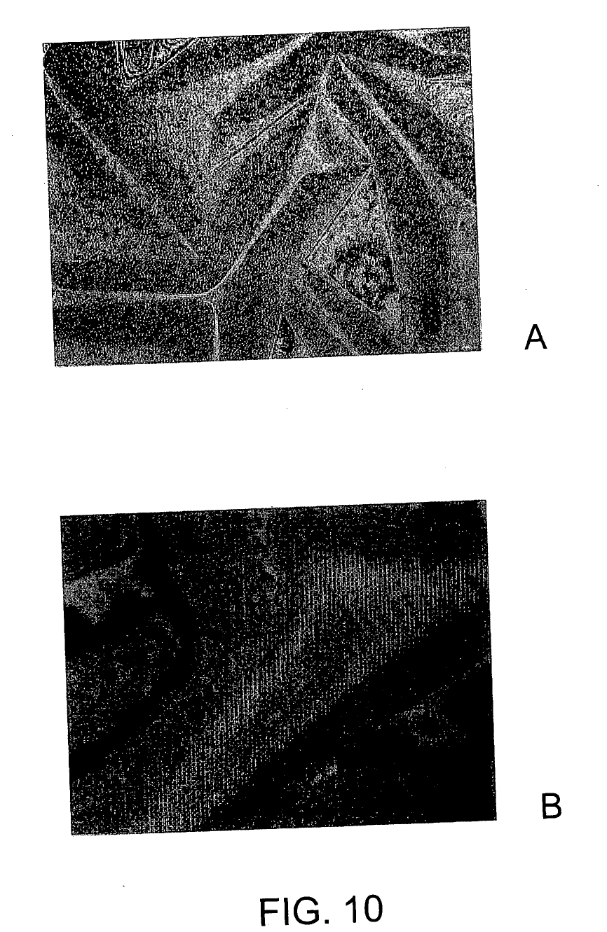

FIG. 10 shows two photographs taken with an optical microscope of a membrane as obtained at the end of step e) of the method according to the invention in a third embodiment of said method in which layer B has been partially liberated from plate A by dry isotropic etching of said plate by means of a plasma (in a “downstream” or “after-glow” type reactor and in a fluorinated chemistry in the presence of oxygen), said isotropic etching having been carried out through the pores formed in said layer B.

Part A of FIG. 10 corresponds to a 1000×enlargement, whereas part B of said Figure corresponds to a 200×enlargement.

Said membrane has a surface area of around 10 mm², a thickness of 100 nm and pores of 350 nm diameter separated from each other by 700 nm.

Said membrane has been formed by using a plate A in silicon and layers B and C in silicon oxide.

Part A of FIG. 10 makes it possible to distinguish the matrix of pores of the membrane, whereas part B of said figure highlights the flexibility of said membrane, the iridescent portions corresponding to the zones where said membrane is hugging the underlying silicon plate.

### Documents Cited

(1) LI et al., Nature, 412, 166-169, 2001.

(2) WO-A-01/36321.

(3) MARTIN and GROVE, Biochemical Microdevices, 3(2), 97-108, 2001.

1. Method for producing a biomimetic membrane (10), characterised in that it comprises the following steps:
a) depositing, on at least one of the principal faces of a plate A of a micro-machinable material, a layer B comprising one or several strata each formed of a micro-machinable material,
b) forming one or several through holes (20) within layer B, each hole having a wall (21) formed of the material(s) of said layer B and a bottom (22) formed of the material of plate A,
c) depositing, on said layer B, the wall (21) and the bottom (22) of each hole, a layer C of a micro-machinable material, which closely hugs the wall and the bottom of said hole,
d) eliminating layer C from the underlying face of layer B and, at the centre of each hole, from the underlying face of plate A, while at the same time leaving a residue (23) of layer C on the wall of said hole(s), said residue delimiting a pore (24) in which the wall (25) is formed of the material of layer C and in which the bottom (26) is formed of the material of layer A, and
e) liberating at least the part of layer B in which are found one or several pores (24) formed in step d), by the partial or total elimination of plate A.

2. Method according to any of the previous claims, characterised in that layer B has a thickness of between around 5 nm and 5 μm.

3. Method according to claim 1 or claim 2, characterised in that the number of through holes (20) formed within layer B, is between 1 single hole and 100 million holes per mm² of surface area of layer B and, preferably, between 1 single hole and 20 million holes per mm² of surface area of layer B.

4. Method according to any of the previous claims, characterised in that the through hole(s) (20) formed in layer B are substantially cylindrical.

5. Method according to any of the previous claims, characterised in that the through hole(s) (20) formed in layer B are formed by a lithography followed by an etching, preferably dry etching.

6. Method according to any of the previous claims, characterised in that step b) comprises an anisotropic etching of layer C.

7. Method according to claim 6, characterised in that the anisotropic etching of layer C is a reactive ion etching.

8. Method according to any of the previous claims, characterised in that step e) comprises the total elimination of plate A.

9. Method according to any of claims 1 to 7, characterised in that step e) comprises the following steps:
e₁) fastening, on the free face of layer B, a plate A′ of a micro-machinable material, and
e₂) hollowing out plates A and A′ so as to liberate the part of layer B in which are found the pore(s), while at the same time leaving the edges of said plates as well as a part of their face opposite to that situated in contact with said layer B.

10. Method according to claim 9, characterised in that plates A and A′ are formed of the same material and are covered, on their face opposite to that situated in contact with layer B, with a layer D or micro-machinable material.

11. Method according to claim 9 or 10, characterised in that step e₂) comprises:
a lithography followed by a wet or dry etching to partially eliminate layers D,
a wet etching to hollow out plates A and A′ while at the same time leaving a residue of said plates which covers layer B, and
a dry etching to liberate the part of layer B in which are found one or several pores.

12. Method according to any of the previous claims, characterised in that layer B comprises a single stratum and in that said stratum is formed of a micro-machinable material different to that forming layer C.

13. Method according to any of claims 1 to 11, characterised in that layer B comprises two strata and in that said strata are formed of two micro-machinable materials different to each other and different to that forming layer C.

14. Method according to any of the previous claims, characterised in that the micro-machinable materials forming plates A and A′, layer B and layer C are chosen from among silicon, polycrystalline silicon, silica, silicon oxide and silicon nitride.

15. Method according to any of the previous claims, characterised in that it comprises, after step e), a step of functionalising the wall of the pore(s) (24) and/or the portions of the principal faces of the membrane which are not occupied by said wall.

16. Method according to claim 15, characterised in that the functionalising step comprises a functionalisation of the wall of the pore(s) (24) and a functionalisation of the portions of the principal faces of the membrane which are not occupied by said wall, said functionalisations being different to each other.

17. Biomimetic membrane (10) with one or several through pores (24), characterised in that it is formed of at least two different micro-machinable materials, one of which forms the wall (23) of said pore(s), whereas the other material(s) form the remainder of said membrane.

18. Biomimetic membrane (10) according to claim 17, characterised in that it has a surface area of between around 1 μm² and 1 cm².

19. Biomimetic membrane (10) according to claim 17 or claim 18, characterised in that it has a thickness of between around 5 nm and 5 μm.

20. Biomimetic membrane (10) according to any of claims 17 to 19, characterised in that it has only one pore or a plurality of pores that may reach 100 million pores per mm² of surface area and, preferably, from 1 single pore to 20 million pores per mm² of surface area.

21. Biomimetic membrane (10) according to any of claims 17 to 20, characterised in that the pore(s) (24) that it comprises are substantially cylindrical and have a diameter of between 5 and 500 nm.

22. Biomimetic membrane (10) according to any of claims 17 to 21, characterised in that it is formed of two or three different micro-machinable materials.

23. Biomimetic membrane (10) according to any of claims 17 to 22, characterised in that the materials forming it are chosen from among silicon, polycrystalline silicon, silica, silicon oxide and silicon nitride.

24. Biomimetic membrane (10) according to any of claims 17 to 23, characterised in that it is integral with two chambers (26, 27) which are arranged on either side of said membrane, which have a base, a lateral wall and a wall opposite said base, and in which said base is formed of said membrane, whereas their wall opposite said base is provided with an opening (28, 29).

25. Biomimetic membrane (10) according to claim 24, characterised in that the lateral wall of the chambers (26, 27) and the wall of said chambers that is opposite their base are formed of a micro-machinable material.

26. Biomimetic membrane (10) according to claim 25, characterised in that said micro-machinable material is chosen from among silicon, polycrystalline silicon, silica, silicon oxide and silicon nitride.

27. Biomimetic membrane (10) according to any of claims 17 to 26, characterised in that the wall of the pore(s) bears chemical and/or biochemical functions different to those borne by the portions of its principal faces which are not occupied by said wall.

28. Application of a biomimetic membrane (10) according to any of claims 17 to 27 to performing studies on the dynamic and functional properties of biological membranes.

29. Application of a biomimetic membrane (10) according to any of claims 17 to 27 to the manufacture of biocatalysis microsystems or the detection or dosing of substances.
